(19) United States
(12) Patent Application Publication
(10) Pub. No.: US 2021/0381409 A1
(43) Pub. Date: Dec. 9, 2021

(54) METHOD AND SYSTEM FOR SENSING ENGINE OIL DETERIORATION

(71) Applicants: HYUNDAI MOTOR COMPANY, SEOUL (KR); KIA MOTORS CORPORATION, SEOUL (KR)

(72) Inventors: Se-Kyu OH, Guri-si (KR); Min-Kyu HAN, Hwaseong-si (KR); Hong-Wook LEE, Seongnam-si (KR); Kyung-Ku YEO, Gwangmyeong-si (KR)

(73) Assignees: HYUNDAI MOTOR COMPANY, SEOUL (KR); KIA MOTORS CORPORATION, SEOUL (KR)

(21) Appl. No.: 16/937,190

(22) Filed: Jul. 23, 2020

(30) Foreign Application Priority Data
Jun. 4, 2020 (KR) 10-2020-0067690

(51) Int. Cl. F01M 11/10; G01N 33/28; B60Q 9/00

(52) U.S. Cl. CPC F01M 11/10; G01N 33/2888; F01M 2011/1433; F01M 2011/142; F01M 2011/1473; B60Q 9/00

(57) ABSTRACT

A method and a system for sensing engine oil deterioration are disclosed. The method includes measuring an engine RPM, load (Load), and an engine temperature (Temp), calculating the amount of change in a deterioration factor (f (RPM, Load, Temp) of engine oil per revolution of an engine using the measured engine RPM, load, and the engine oil temperature, and then calculating the engine oil deterioration degree at a current time point by accumulating a value obtained by multiplying the calculated amount in change of the deterioration factor (f (RPM, Load, Temp) by an engine revolutions during a predetermined sampling cycle (Δr) to a previous engine oil deterioration degree.

### CROSS-REFERENCE TO RELATED APPLICATION

The present application claims priority to and the benefit of Korean Patent Application No. 10-2020-0067690, filed on Jun. 4, 2020, which is incorporated herein by reference in its entirety.

### TECHNICAL FIELD

The present disclosure relates to a method and a system for sensing engine oil engine oil deterioration, and more particularly, to a method and a system for sensing engine oil deterioration, which may predict a deterioration state of engine oil, and may be applied to various types of vehicles.

### BACKGROUND

The statements in this section merely provide background information related to the present disclosure and may not constitute prior art.

In the case of the engine oil of a vehicle, the oil deteriorates when used for a long time, and the oil gradually changes from alkaline to acidic with the use of the oil. Meanwhile, since a value of the total base number (TBN) in the oil gradually decreases with the use of the oil, the degree of deterioration of the oil may be accurately known by measuring the TBN. However, the equipment for measuring the TBN is difficult to mount to a vehicle, and thus various technologies have been proposed as alternatives.

As an example, a method for measuring a change in viscosity of the oil using a pressure sensor which measures a pressure difference of an oil passage or a discharge pressure of an oil pump based on the fact that the viscosity increases when the oil deteriorates may be proposed, but this may not necessarily be estimated as the deterioration of the oil according to a difference of pressure and the method is not generalized due to a serious deviation in the measurement.

### SUMMARY

The present disclosure provides a method and a system for sensing engine oil deterioration, which may accurately predict a deterioration state of engine oil in a simple method, and may be applied to various types of vehicles such as an internal combustion engine vehicle and a hybrid vehicle.

A method for sensing engine oil deterioration according to the present disclosure for achieving the object includes: measuring an engine RPM, load, and an engine oil temperature; calculating the amount of change in a deterioration factor (f (RPM, Load, Temp)) of engine oil per revolution of an engine using the measured engine RPM, load, and engine oil temperature; and calculating the engine oil deterioration degree at a current time point by accumulating a value obtained by multiplying engine revolutions during a predetermined sampling cycle (Δr) by the calculated amount of change in the deterioration factor (f (RPM, Load, Temp)) to a previous engine oil deterioration degree.

At this time, the amount of change in the deterioration factor (f (RPM, Load, Temp)) may be calculated by Equation 1 below:

$$f ( RPM , Load , Temp ) = {a}_{0} + {a}_{1} ( \frac{1}{RPM} ) + {a}_{2} ( \frac{1}{Load + 100} ) + {a}_{3} \times Temp + {a}_{4} \times {Temp}^{2} \qquad Equation 1$$

where RPM refers to the engine RPM, Load refers to engine load (kgf·m), Temp refers to the engine oil temperature (° C.), and a₀, a₁, a₂, a₃, and a₄ refer to coefficients.

Meanwhile, the method may further include: displaying an engine oil change warning to a driver, if the accumulated engine oil deterioration degree at the current time point is a predetermined value or more.

Alternatively, the method may operate the residual oil life based on the oil deterioration degree, and also display relevant information to the driver according to the operated residual oil life. To this end, preferably, the method may further include: operating a residual life of the engine oil using a predetermined reference maximum deterioration degree index value, and the accumulated engine deterioration degree at the current time point; and displaying an engine oil change warning to a driver, if the operated residual life is a predetermined value or less.

In addition, preferably, the method may further include: operating the residual life of the engine oil based on at least any one of a traveling time of a vehicle or a traveling distance of the vehicle so as to supplement a method for calculating the residual oil life based on the oil deterioration degree, and may determine a minimum value as the residual life of the engine oil by comparing the residual life of the engine oil operated using the engine oil deterioration degree at the current time point with the residual life of the engine oil operated based on at least any one of the traveling time of the vehicle or the traveling distance of the vehicle, and also display the engine oil change warning to the driver, if the determined residual life of the engine oil is the predetermined value or less.

Meanwhile, preferably, the engine oil deterioration degree may be stored in an EEPROM of an engine control unit so as not to be deleted even when the engine is turned off.

In addition, the engine oil deterioration degree may be reset as a predetermined initial oil determination degree value (X(0)) by operating a diagnostic device or an input button of a cluster when the engine oil is changed.

A system for sensing engine oil deterioration according to the present disclosure for achieving the object includes: an engine information collection unit which measures an engine RPM (RPM), load (Load), and an engine oil temperature (Temp); a deterioration factor calculation unit which calculates the amount of change in a deterioration factor (f (RPM, Load, Temp)) of engine oil per revolution of an engine using the engine RPM, load, and engine oil temperature collected by the engine information collection unit; and a residual oil life operation unit which calculates a residual oil life using the amount of change in the deterioration factor calculated by the deterioration factor calculation unit, in which the residual oil life operation unit calculates the engine oil deterioration degree at a current time point by accumulating a value obtained by multiplying engine revolutions during a predetermined sampling cycle (Δr) by the calculated amount of change in the deterioration factor to a previous engine oil deterioration degree.

Preferably, the system for sensing the engine oil deterioration may further include: a display unit which sends an oil change alarm according to the operation result of the residual oil life operation unit.

In addition, preferably, the residual oil life operation unit may further include: at least any one of an oil change mileage-based residual oil life operation unit which operates the residual oil life based on a traveling distance of a vehicle, and an oil change cycle-based residual oil life operation unit which operates the residual oil life based on a traveling time of the vehicle, and the system for sensing the engine oil deterioration may further includes a residual oil life determination unit which determines, as the residual oil life, a minimum value of the residual oil life calculated using the amount of change in the deterioration factor calculated by the deterioration factor calculation unit, and the residual oil lives each operated by the oil change mileage-based residual oil life operation unit and the oil change cycle-based residual oil life operation unit.

According to the method and system for sensing the engine oil deterioration according to the present disclosure, by using the deterioration factor confirmed to increase linearly according to the increase in the engine revolutions under the same operating conditions (RPM, load, oil temperature), it is possible to accurately predict the current oil state.

According to the method and system for sensing the engine oil deterioration according to the present disclosure, by calculating the residual oil life by accumulating the oil deterioration degree based on the engine revolutions, it(the method and system for sensing the engine oil deterioration is applicable regardless of the idling cycle of the engine, and it is possible to accurately predict the oil state not only for the internal combustion engine vehicle but also for the intermittently engine-operated hybrid vehicle, in addition to the general internal combustion engine.

In addition, according to the method and system for sensing the engine oil deterioration according to the present disclosure, it is possible to predict the oil deterioration degree in real time using the operating conditions even without the separate additional measurement equipment, and notify the driver of the predicted oil change time point, thereby preventing the engine damage caused by not changing the engine oil, and preventing the engine oil from being unnecessarily changed frequently.

Further areas of applicability will become apparent from the description provided herein. It should be understood that the description and specific examples are intended for purposes of illustration only and are not intended to limit the scope of the present disclosure.

### DRAWINGS

In order that the disclosure may be well understood, there will now be described various forms thereof, given by way of example, reference being made to the accompanying drawings, in which:

FIG. 1 is a block diagram illustrating a configuration of a system for sensing engine oil deterioration in one form of the present disclosure.

FIG. 2 is a diagram illustrating the flow for calculating the oil deterioration degree in the method and the system for sensing the engine oil deterioration in one form of the present disclosure.

FIG. 3 is a flowchart illustrating the method for sensing the engine oil deterioration in one form of the present disclosure.

The drawings described herein are for illustration purposes only and are not intended to limit the scope of the present disclosure in any way.

### DETAILED DESCRIPTION

The following description is merely exemplary in nature and is not intended to limit the present disclosure, application, or uses. It should be understood that throughout the drawings, corresponding reference numerals indicate like or corresponding parts and features.

The technology of measuring the capacitance by mounting an electrode to measure the amount of change in a polar group caused by the deterioration of the oil was presented in the past, but in this case, there are various factors affecting the capacitance, and the amount of change is also very small, so there is a measurement error limitation of the measurement value, thereby being not suitable for being mounted to the actual vehicle.

Alternatively, the technology of measuring the absorbance of engine oil using near infrared rays having a wavelength of 1000 nm or more, and determining a deterioration state of the engine oil according to an increase rate of the absorbance has been introduced.

However, it also needs to provide a separate infrared sensor capable of irradiating near-infrared rays and a system for converting the near-infrared rays into an electrical signal separately, the features of the absorbance may vary depending on the type of oil supplied, and it may not be easy to response to various types of oils because data of the initial absorbance is required for the various oil.

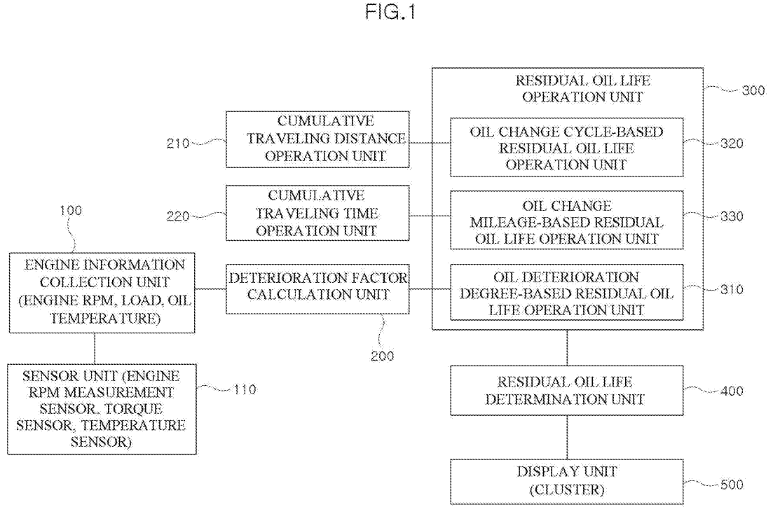

FIG. 1 is a block diagram illustrating a configuration of a system for sensing engine oil deterioration according to a preferred exemplary embodiment of the present disclosure.

As illustrated in FIG. 1, a system for sensing engine oil deterioration according to a preferred exemplary embodiment of the present disclosure basically includes an engine information collection unit 100 which measures an engine RPM (RPM), load (Load), and an engine oil temperature (Temp), a deterioration factor calculation unit 200 which calculates the amount of change in a deterioration factor (f (RPM, Load, Temp)) of engine oil per revolution of an engine using the engine RPM, the load, and the engine oil temperature collected by the engine information collection unit 100, and a residual oil life operation unit 310 based on the oil deterioration degree which calculates the residual oil life using the amount of change in the deterioration factor calculated by the deterioration factor calculation unit 200.

The engine information collection unit 100 performs a function of collecting the engine RPM, the load, and the engine oil temperature through a sensor unit 110, and transferring them to the deterioration factor calculation unit 200.

Here, the sensor unit 110 is composed of a plurality of sensors for sensing the vehicle speed, the engine RPM, the engine oil temperature, a throttle valve opening, and the like, respectively.

Preferably, the engine RPM may be collected through an engine RPM measurement sensor for measuring the engine RPM by generating a predetermined signal each time a crankshaft rotates, such as a hall sensor.

The load may be measured directly from a torque sensor, or by sensing the engine RPM and the amount of opening of the throttle valve, an electronic control unit of a vehicle may also calculate the load according to the sensed result through a modeling.

In addition, the engine oil temperature may be measured by a temperature sensor installed on a circulation circuit of the engine oil.

The information about the engine RPM, the load, and the engine oil temperature collected by the engine information collection unit 100 is transferred to the deterioration factor calculation unit 200 to be used to calculate the amount of change in the deterioration factor (X) of the engine oil per revolution of the engine.

The deterioration of the engine oil is affected by the engine RPM, the load, and the engine oil temperature. In addition, the engine oil deterioration occurs due to the generation of nitride and oxide. Accordingly, the present disclosure sets the deterioration factor (X) for indicating the deterioration state of the oil to an average value of oxidation determined by the amount of oxide contained in the engine oil and nitration determined by the amount of nitride. In addition, the amount of change in the deterioration factor (X) per revolution of the engine is set to a value expressed by a function f (RPM, Load, Temp) related to the engine RPM, the load, and the engine oil temperature. Specifically, the amount of change in the deterioration factor (X) per revolution of the engine is calculated using the information about the engine RPM, the load, and the engine oil temperature collected by the engine information collection unit 100, and Equation 1 below.

$$f ( RPM , Load , Temp ) = {a}_{0} + {a}_{1} ( \frac{1}{RPM} ) + {a}_{2} ( \frac{1}{Load + 100} ) + {a}_{3} \times Temp + {a}_{4} \times {Temp}^{2} \qquad Equation 1$$

Where RPM refers to the engine RPM, Load refer to the load (kgf·m), Temp refers to the engine oil temperature (° C.), and a₀, a₁, a₂, a₃, a₄ are coefficients.

Equation 1 is obtained through data analysis on the relationship between the oxidation and nitration related to the degree of the oil deterioration, the engine RPM, the load, and the engine oil temperature, where the coefficients a₀, a₁, a₂, a₃, a₄ are obtained by approximating (fitting) test data using the least squares method. For the same engine, the values of the five coefficients are fixed as constants.

The residual oil life operation unit 300 calculates the degree of the engine oil deterioration at the current time point by accumulating a value obtained by multiplying the amount of change in the deterioration factor (X) per revolution of the engine calculated using Equation 1 in the deterioration factor calculation unit 200 by the engine revolutions during a predetermined sampling cycle (Δr) to the previous degree of the engine deterioration.

That is, the method for sensing the engine oil deterioration according to the present disclosure does not accumulate the above value based on time but accumulates the above value based on the engine revolutions, in calculating the degree of the oil deterioration using the deterioration factor (X). This is because the engine oil mainly deteriorates in the process of lubrication of the engine, and thus for the oil deterioration, how many the engine has actually rotated is more important than an elapsed time after the engine oil is changed or a travel distance.

Meanwhile, according to the research of the inventors of the present disclosure, it is shown that the deterioration factor (X), which is the average value of the oxidation and nitration of the oil, changes linearly with the change in the engine revolutions under the same operating conditions (RPM, load, and oil temperature).

Accordingly, in the method for sensing the engine oil deterioration according to the present disclosure, the change in the deterioration factor (X) for determining the oil deterioration degree is expressed in the form of a differential equation which is changed based on the engine revolutions (r), not the time, as expressed in Equation 2 below.

$$\frac{dX ( r )}{dr} = f ( RPM , Load , Temp ) \qquad Equation 2$$

Meanwhile, in order to perform the calculation in an engine control unit (ECU) of the vehicle, Equation 3 below is obtained by discretizing Equation 2. Where Δr refers to the amount of change (increase) in the engine revolutions during a predetermined sampling interval.

$$\frac{X ( r + Δ r ) - X ( r )}{Δ r} = f ( RPM , Load , Temp ) \qquad Equation 3$$

Summarizing Equation 3 so that the deterioration factor value (X(r+Δr)) when the engine is rotated by Δr is moved to the left side, Equation 4 below is obtained.

X(r+Δr)=X(r)+Δr×f(RPM,Load,Temp) Equation 4

According to Equation 4, the deterioration factor value (X(r+Δr)) when the engine is rotated by Δr calculates the engine oil deterioration degree at the current time point by accumulating a value obtained by multiplying the engine revolutions during a predetermined sampling cycle (Δr) by the amount of change in the deterioration factor (X) per revolution of the engine to the previous degree of engine deterioration (X(Δr)). In addition, an initial value of the oil deterioration degree value (X(0)) is set as a predetermined value obtained by operating a diagnostic device or a cluster menu of the vehicle when the engine oil is initially injected and each time the engine oil is changed, and input to an EEPROM of the engine control unit.

For example, if a sampling time is set as 1 second and an operation per second is performed, the engine revolutions for 1 second (Δr), which is the sampling time, may be expressed by Equation 5 below. Where k means a cumulative time (s).

Δr=r(k+1)−r(k) Equation 5

In addition, as expressed in Equation 6, the deterioration factor value (X(k+1)) after (k+1) seconds is calculated as a value obtained by multiplying the deterioration factor value X(k) before k seconds by the engine revolutions for 1 second (Δr) and the amount of change in the deterioration factor (X) per revolution of the engine (f (RPM, Load, Temp)).

X(k+1)=X(k)+Δr×f(RPM,Load,Temp) Equation 6

As described above, if the sampling time is 1 second, the engine oil deterioration degree is calculated every 1 second.

However, since the oil deterioration proceeds relatively slowly, it is not necessary to perform the operation per revolution of the engine. That is, in order to calculate the oil deterioration degree, it is necessary to perform the operation every specific sampling cycle, and the sampling cycle may be appropriately set according to the operation capability of the engine control unit.

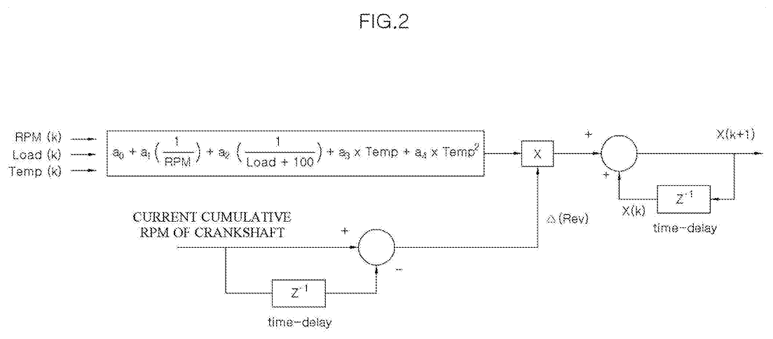

FIG. 2 illustrates the flow of a method for calculating the oil deterioration degree performed by the residual oil life operation unit 400 using the deterioration factor calculated by the deterioration factor calculation unit 200 in connection with the aforementioned example in which the sampling time is 1 second.

As illustrated in FIG. 2, when the information about the engine RPM (RPM), the load (Load), and the engine oil temperature (Temp) at k seconds is collected from the engine information collection unit 100, the amount of change in the deterioration factor (X) per revolution of the engine at k seconds is calculated using the corresponding information.

In addition, as illustrated in FIG. 2, when the cumulative revolutions of the crankshaft at the current time point ((k+1) seconds) passes through a time-delay box, it becomes the cumulative revolutions 1 second ago, and when the cumulative revolutions of the crankshaft 1 second ago is subtracted from the current cumulative revolutions of the crankshaft, the engine revolutions (Δr) of the crankshaft per 1 second is calculated. In addition, the deterioration factor (X(k)) at (k+1) seconds is calculated by adding the deterioration factor ((X(k)) value at k seconds to a value obtained by multiplying the amount in change in the deterioration factor (X) per revolution of the engine at k seconds by the engine revolutions of the crankshaft per 1 second (Δr). Meanwhile, the oil deterioration factor (the oil deterioration degree) is preferably stored in the EEPROM of the engine control unit (ECU) so as not to be erased even when the engine is turned off. In addition, when the engine oil is changed, the value of the oil deterioration degree is reset through the operation of the diagnostic device or the operation of the cluster menu of the vehicle.

When the deterioration factor (X(k+1)) value at the current time point is calculated, the oil deterioration degree-based residual oil life operation unit 310 operates the residual oil life of the engine oil using the deterioration factor (X).

Preferably, the oil deterioration degree-based residual oil life operation unit 310 may determine a case where the deterioration factor (X(k+1)) value at the current time point is a predetermined value or more as the time point at which the engine oil is to be changed. Accordingly, in this case, as described later, the residual oil life operation unit 310 may cause a display unit 500 to display an engine oil change warning to a driver.

Meanwhile, the oil deterioration degree-based residual oil life operation unit 310 stores a predetermined reference maximum deterioration degree index value, and may predict a residual life through a ratio of the reference maximum deterioration degree index value and the deterioration factor value (X(k+1)) at the current time point. Accordingly, when the deterioration factor value (X(k+1)) at the current time point reaches a certain ratio or more with respect to the reference maximum deterioration degree index value, the residual oil life operation unit 310 may cause the display unit 500 to display the engine oil change warning to the driver. In addition, even if the deterioration factor value (X(k+1)) at the current time point does not reach the certain ratio, for the purpose of information transfer, the residual oil life operation unit 310 may cause the display unit 500 to display the estimated residual life of the current engine oil.

Meanwhile, as illustrated in FIG. 1, the residual oil life operation unit 300 may further include an oil change cycle-based residual oil life operation unit 320 and/or an oil change mileage-based residual oil life operation unit 330, in addition to the oil deterioration degree-based residual oil life operation unit 310 described above in order to predict the residual oil life.

The oil change cycle-based residual oil life operation unit 320 calculates a cumulative traveling time of the vehicle after changing the engine oil through the runtime operation of the engine control unit (ECU), and predicts the residual oil life by comparing the cumulative traveling time with a predetermined maximum oil change time cycle.

The oil change mileage-based residual oil life operation unit 330 calculates a cumulative traveling distance of the vehicle after changing the engine oil through the runtime operation of the engine control unit (ECU), and predicts the residual oil life by comparing the cumulative traveling distance with a predetermined maximum oil change mileage.

A residual oil life determination unit 400 determines a final residual oil life based on the operation result from the residual oil life operation unit 300.

Preferably, the residual oil life determination unit 400 may determine the residual life calculated by the oil deterioration degree-based residual oil life operation unit 310 as the final residual oil life.

Alternatively, if the residual oil life operation unit 300 further includes the oil change cycle-based residual oil life operation unit 320 and/or the oil change mileage-based residual oil life operation unit 330 in addition to the oil deterioration degree-based residual oil life operation unit 310, the residual oil life determination unit 400 determines the final residual life of the residual life corresponding to a minimum value by comparing the residual lives each operated by the oil deterioration degree-based residual oil life operation unit 310, the oil change cycle-based residual oil life operation unit 320 and/or the oil change mileage-based residual oil life operation unit 330 with each other.

In the case of the vehicle having a long idling cycle or a hybrid vehicle in which the engine operates intermittently, the prediction of the residual life using the oil change cycle or the oil change mileage may be inaccurate. Accordingly, the residual oil life determination unit 400 preferably determines, as the final residual life, the smallest value among the residual lives each operated by the oil deterioration degree-based residual oil life operation unit 310, the oil change cycle-based residual oil life operation unit 320, and/or the oil change mileage-based residual oil life operation unit 330.

When the final residual life is determined, the residual oil life determination unit 400 allows the display unit 500 to notify the driver of an alarm in connection to the determined residual life.

At this time, the residual oil life determination unit 400 allows the display unit 500 not only to notify when the oil change time arrives, but also to divide the oil change time into several steps to be predictable to perform the alarm. That is, by dividing the oil change time into a primary time and a secondary time, it is possible to alert the driver of the corresponding fact every time the determined residual life reaches the respective warning life reference values.

The display unit 500 performs a function of alerting when the change time of the engine oil arrives based on the engine oil deterioration degree sensed by the method for sensing the engine oil deterioration according to the present disclosure. In addition, as described above, it is also possible to perform a function of guiding the residual life and the change time of the engine oil step by step. Preferably, the display unit 500 may be a cluster installed on a driver seat, and related information may be notified by a pop-up on the cluster.

Meanwhile, each of the components such as the engine information collection unit 100, the deterioration factor calculation unit 200, the residual oil life operation unit 300, and the residual oil life determination unit 400 configuring the system for sensing the engine oil deterioration may also be realized in the form of a computer provided in the vehicle. In that case, the program for realizing this control function may be recorded on a computer-readable recording medium, and the program recorded on the recording medium may be realized by being read and executed in a computer system. In addition, the “computer system” referred to herein is a computer system embedded in the vehicle, and is assumed to include hardware such as an OS or peripheral devices. In addition, the term “computer-readable recording medium” refers to a storage device such as a flexible disk, an optical magnetic disk, a portable medium such as a ROM or a CD-ROM, or a hard disk embedded in the computer system. In addition, the term “computer-readable recording medium” means that the program is held for a short time and dynamically, such as a communication line in the case of transmitting a program through a network such as the Internet or a communication line such as a telephone line, in which case the program is also held for a certain time such as a volatile memory inside the computer system to be used as a server or a client. In addition, the program may be for realizing a portion of the aforementioned functions, or may be implemented in combination with a program in which the aforementioned function is already recorded in the computer system.

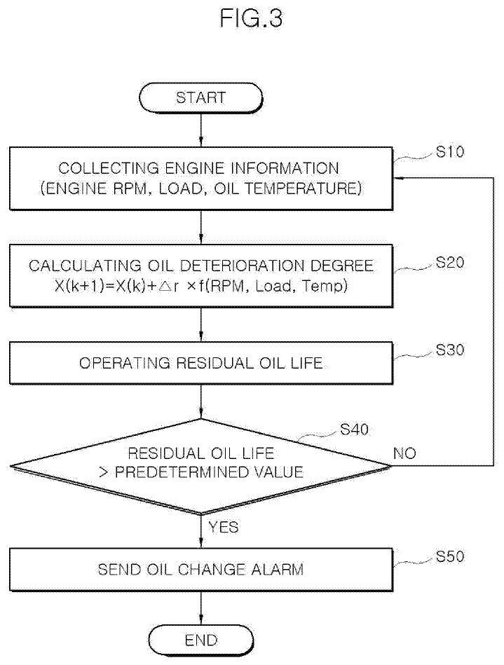

Hereinafter, a preferred exemplary embodiment of the method for sensing the engine oil deterioration according to the present disclosure will be described in detail with reference to FIG. 3. FIG. 3 is a flowchart illustrating a method for sensing the engine oil deterioration according to a preferred exemplary embodiment of the present disclosure.

When the initial injection of the engine oil is completed, or when the engine oil is changed, the oil deterioration factor is reset to a predetermined initial value (X(0)) by operating the diagnostic device or the cluster menu of the vehicle. The engine oil deterioration degree by the method for sensing the engine oil deterioration illustrated in FIG. 3 starts to be sensed from that time.

To this end, first, collecting engine information (S10) is performed which collects, by the engine information collection unit 100, related information by measuring the engine RPM, the load (Load), and the engine oil temperature (Temp).

In addition, the deterioration factor calculation unit 200 calculates the engine oil deterioration degree using the engine RPM, the load, and the engine oil temperature collected in the S10 (S20). To this end, the deterioration factor calculation unit 200 first calculates the amount of change in the deterioration factor of the engine oil per revolution of the engine (f(RPM, Load, Temp)). In addition, the engine information collection unit 100 is used to collect the information about the engine revolutions which occurs during a predetermined sampling cycle (for example, 1 second). In addition, the engine oil deterioration degree (the amount of change in the deterioration factor) is calculated during the predetermined sampling cycle using the amount of change in the deterioration factor of the engine oil per revolution of the engine and the engine revolutions generated during the predetermined sampling cycle, and the current oil deterioration degree value (X(k+1)) is calculated by accumulating the engine oil deterioration degree to the previous deterioration degree value (X(k)).

Since the specific calculation methods of the amount of change in the deterioration factor of the engine oil per revolution of the engine and the oil deterioration degree using the same have been described above in detail, a duplicate description is omitted.

When the engine oil deterioration degree is calculated, the residual oil life operation unit 300 and the residual oil life determination unit 400 calculate and determine the residual oil life.

Preferably, when it is shown that the current engine oil deterioration degree value (X(k+1)) calculated in the S20 exceeds a predetermined reference value, it may be determined that the life of the corresponding engine oil is exhausted, and in this case, the display unit 500 may be controlled to display the engine oil change warning to the driver.

Alternatively, when the current engine oil deterioration degree value (X(k+1)) calculated in the S20 exceeds the predetermined reference value, the residual life may be operated through the ratio of the comparison result by comparing the current engine oil deterioration degree value (X(k+1)) with a predetermined reference maximum deterioration degree index value (S30).

In addition, by comparing the operated residual oil life with a predetermined value (S40), when the residual oil life is less than the predetermined value, that is, the deterioration factor value (X(k+1)) at the current time point reaches a certain ratio or more with respect to the reference maximum deterioration degree index value, the display unit 500 is controlled to send an oil change alarm to the driver (S50).

Meanwhile, preferably, as described above, in order to supplement the method for calculating the residual oil life based on the engine oil deterioration degree, the method may further include operating the residual life of the engine oil based on at least any one of the traveling time of the vehicle or the traveling distance of the vehicle. In this case, in the S30, by comparing the residual life of the engine oil operated using the engine oil deterioration degree at the current time point with the residual life of the engine oil operated based on at least any one of the traveling time of the vehicle or the traveling distance of the vehicle, the minimum value is determined as the residual life of the engine oil so as to become the residual oil life to be compared with the predetermined value in the S40.

The description of the disclosure is merely exemplary in nature and, thus, variations that do not depart from the substance of the disclosure are intended to be within the scope of the disclosure. Such variations are not to be regarded as a departure from the spirit and scope of the disclosure.

What is claimed is:

1. A method for sensing engine oil deterioration, the method comprising:
measuring an engine revolutions per minute (RPM), a load, and an engine oil temperature;
calculating an amount of change in a deterioration factor (f (RPM, Load, Temp)) of engine oil per revolution of an engine using the measured engine RPM, the measured load, and the measured engine oil temperature; and
calculating an engine oil deterioration degree at a current time point by accumulating a value obtained by multiplying an engine revolutions during a predetermined sampling cycle (Δr) by the calculated amount of change in the deterioration factor (f (RPM, Load, Temp)) to a previous engine oil deterioration degree.

2. The method of claim 1, wherein the amount of change in the deterioration factor (f (RPM, Load, Temp)) is calculated by Equation 1 below:
$$f ( RPM , Load , Temp ) = {a}_{0} + {a}_{1} ( \frac{1}{RPM} ) + {a}_{2} ( \frac{1}{Load + 100} ) + {a}_{3} \times Temp + {a}_{4} \times {Temp}^{2}$$
where RPM refers to the engine RPM, Load refers to engine load (kgf·m), Temp refers to the engine oil temperature (° C.), and a₀, a₁, a₂, a₃, and a₄ refer to coefficients.

3. The method of claim 1, wherein the method further comprises:
displaying an engine oil change warning to a driver when the accumulated engine oil deterioration degree at the current time point is greater than or equal to a predetermined value.

4. The method of claim 1, wherein the method further comprises:
operating a residual life of the engine oil using a predetermined reference maximum deterioration degree index value, and the accumulated engine oil deterioration degree at the current time point; and
displaying an engine oil change warning to a driver when the operated residual life is less than or equal to a predetermined value.

5. The method of claim 4, wherein the method further comprises:
operating the residual life of the engine oil based on at least one of a traveling time of a vehicle or a traveling distance of the vehicle;
determining that a minimum value is the residual life of the engine oil by comparing the residual life of the engine oil using the engine oil deterioration degree at the current time point with the residual life of the engine oil based on at least one of the traveling time of the vehicle or the traveling distance of the vehicle; and
displaying the engine oil change warning to the driver when the determined residual life of the engine oil is less than or equal to the predetermined value.

6. The method of claim 1, wherein the method comprises:
storing the engine oil deterioration degree in an electrically erasable programmable read-only memory (EEPROM) of an engine control unit.

7. The method of claim 1, wherein the method comprises:
resetting the engine oil deterioration degree by operating a diagnostic device or an input button of a cluster when the engine oil is changed.

8. A system for sensing engine oil deterioration comprising:
an engine information collection unit configured to measure an engine RPM (RPM), load (Load), and an engine oil temperature (Temp);
a deterioration factor calculation unit configured to calculate an amount of change in a deterioration factor (f (RPM, Load, Temp)) of engine oil per revolution of an engine using the engine RPM, the load, and the engine oil temperature; and
a residual oil life operation unit configured to:
calculate a residual oil life using the calculated amount of change in the deterioration factor; and
calculate the engine oil deterioration degree at a current time point by accumulating a value obtained by multiplying an engine revolutions during a predetermined sampling cycle (Δr) by the calculated amount of change in the deterioration factor to a previous engine oil deterioration degree.

9. The system of claim 8, wherein the system further comprises:
a display unit configured to display an oil change alarm according to an operation result of the residual oil life operation unit.

10. The system of claim 9, wherein the residual oil life operation unit further comprises at least one of an oil change mileage-based residual oil life operation unit configured to operate the residual oil life based on a traveling distance of a vehicle, or an oil change cycle-based residual oil life operation unit configured to operate the residual oil life based on a traveling time of the vehicle, and
wherein the system further comprises:
a residual oil life determination unit configured to determine that a minimum value of the residual oil life calculated using the amount of change in the deterioration factor calculated by the deterioration factor calculation unit is the residual oil life.
